(19) United States
(12) Patent Application Publication
(10) Pub. No.: US 2008/0154157 A1
(43) Pub. Date: Jun. 26, 2008

(54) COSMETIC AND BIOMEDICAL APPLICATIONS OF ULTRASONIC ENERGY AND METHODS OF GENERATION THEREOF

(75) Inventors: Gregory B. Altshuler, Lincoln, MA (US); Mikhail Smirnov, Burlington, MA (US); IIya Yaroslavsky, North Andover, MA (US); Pavel Kamaev, North Reading, MA (US); Andrei V. Erofeev, North Andover, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: PALOMAR MEDICAL TECHNOLOGIES, INC., Burlington, MA (US)

(21) Appl. No.: 11/956,027

(22) Filed: Dec. 13, 2007

Related U.S. Application Data
(60) Provisional application No. 60/874,606, filed on Dec. 13, 2006.

(51) Int. Cl. A61N 7/00

(52) U.S. Cl. 601/2

(57) ABSTRACT

Methods and devices are disclosed for treatment of tissue, such as skin tissue, using acoustic energy, such as ultrasound. The ultrasound can be used for various purposes include hair removal and permanent hair reduction, removal of tattoos, and other cosmetic procedures. In some embodiments, ultrasound is produced using an optical-to-acoustic converter, and, in some embodiments, ultrasound is focused.

### RELATED APPLICATIONS

This application claims the benefit of U.S. Provisional Application No. 60/874,606, filed Dec. 13, 2006, which is incorporated herein by reference in its entirety.

### BACKGROUND OF THE INVENTION

1. Field of the Invention

The devices and methods disclosed herein relate to the treatment of soft and hard tissues with acoustic energy generally, including ultrasonic energy, to stimulate and facilitate repair and healing in a controlled fashion. The devices and methods also relate to systems for treating in hard and soft tissue using ultrasound, and cosmetic, medical and other applications of such devices, methods and systems.

2. Description of the Related Art

Various techniques (mechanical, chemical, light-induced, etc.) for managing hair growth and depilation are known in the art, as well as for other cosmetic treatments. For example, the use of high-intensity ultrasonic energy for treating various cutaneous and sub-cutaneous conditions has been disclosed in the following: PCT Publication WO 00/21612 entitled “A method and device for hair removal;” U.S. Pat. No. 6,544,259 entitled “Hair removal method and device;” U.S. Pat. No. 5,346,499 entitled “Depilation apparatus and method using a vibration member to affect the function of nerves in the skin;” U.S. Pat. No. 6,113,559 entitled “Method and apparatus for therapeutic treatment of skin with ultrasound;” U.S. Pat. No. 6,595,934 entitled “Methods of skin rejuvenation using high-intensity focused ultrasound to form an ablated tissue area containing a plurality of lesions.” However, these and other existing attempts to use ultrasound for cosmetic and other purposes have not yielded practical and functional techniques that effectively generate and apply ultrasound for their intended uses.

### SUMMARY OF THE INVENTION

The inventors have developed a more effective method of treating tissue using ultrasound or other acoustic energy.

One aspect of the invention is a device for treating a tissue with acoustic energy. The device can include a source of electromagnetic energy and an energy absorption medium configured to accept electromagnetic energy generated by the source. The device can also include a transduction member configured to transduce electromagnetic energy to acoustic energy and further configured to receive energy from the absorption medium. The device can also include a focusing element in communication with the transduction member and configured to focus the acoustic energy and direct the acoustic energy to a tissue during operation.

Preferred embodiments of this aspect of the invention can include one or more of the following. The transduction member can include a liquid substance configured to convert optical energy to ultrasound or it can include a solid member configured to convert the electromagnetic energy to ultrasound. The transduction member can also include a single piezoelectric element or an array of piezoelectric elements. The focusing element can be a concave surface configured to transmit the acoustic energy, can be an optical element or can be another type of structure that allows the resulting acoustic energy to be concentrated.

Another aspect of the invention is a device for treating a tissue with acoustic energy. The device can have a source of electromagnetic energy, a converter and an output. The converter can be configured to receive electromagnetic energy from the source and convert the electromagnetic energy to acoustic energy. The output can be in communication with the converter and configured to transmit the acoustic energy to a tissue.

Preferred embodiments of this aspect of the invention can include one or more of the following. The acoustic energy can be ultrasound. The converter can include a piezoelectric element, such as a piezoceramic element or other piezoelement. The piezoelectric element can be configured to focus the acoustic energy. The converter can also include an array of piezoelectric elements, and the array of piezoelectric elements can be configured to focus the acoustic energy. The device can have another type of focusing element configured to focus the acoustic energy. The focusing element can be included in the converter or in the output or in another location.

Another aspect of the invention is a device for treating a tissue with acoustic energy that includes a source of optical radiation, an absorbing member, a converter, and an output. The absorbing member can be configured to absorb energy from the source. The converter can be configured to receive the absorbed energy from the absorbing member and to convert the energy to focused ultrasonic. The output can be in communication with the converter and configured to deliver the ultrasonic energy to a tissue.

Another aspect of the invention is a method of treating tissue with acoustic energy comprising irradiating a fist medium with electromagnetic radiation; converting the electromagnetic radiation to acoustic energy; focusing the acoustic energy; and delivering the acoustic energy to a tissue to be treated.

Preferred embodiments of this aspect of the invention can include one or more of the following. The acoustic energy can be ultrasound. The acoustic energy can be focused into a single beam, or into an array of beams. The acoustic energy can be delivered to a portion of the tissue that selectively absorbs the acoustic energy.

Another aspect of the invention is a method of treating tissue with acoustic energy comprising irradiating a fist medium with electromagnetic radiation, converting the electromagnetic radiation to acoustic energy, and delivering the acoustic energy to the tissue to be treated. The acoustic energy can have at least one frequency component that is resonant with a structure in a tissue to be treated.

Preferred embodiments of this aspect of the invention can include one or more of the following. The structure in the tissue can be a portion of a hair or a portion of a hair follicle. The acoustic energy can be focused. The acoustic energy can be ultrasound.

The embodiments discussed below provide devices and methods for generating ultrasonic energy, delivering it to tissue and applying it for various cosmetic and other treatments, including (but not limited to) treatment of cellulite; improvement of skin appearance, tone, and/or texture; complete or partial removal of tattoos; and reducing the rate of hair growth or for hair removal. Some embodiments generate relatively low-power ultrasonic energy, with a lower cost energy source, and do not require cooling of the tissue being treated. Such devices present relatively few safety concerns and the performance is not dependent on the level of pigmentation in the tissue. Other embodiments have higher intensity and can benefit from cooling to protect skin or lessen the pain of such treatments (or both).

In other embodiments, treatments and devices using selective sonothermolysis are also disclosed. Some of the possible embodiments have the advantage of eliminating the need to use high-power electromagnetic radiation in a treatment device. Thus, concerns regarding energy levels, skin damage, eye safety, device cost, treating a wide range of skin pigmentations, and cooling requirements can be eliminated or reduced.

### BRIEF DESCRIPTION OF THE FIGURES

FIG. 1 is a side cross-sectional schematic view of a laser-based ultrasonic system used for the treatment of tissue.

FIG. 2 is a graph illustrating the measured laser impulse as a function of time for the system of FIG. 1.

FIG. 3 is a graph illustrating the energy produced by the system of FIG. 1 as a function of pumping voltage, in the case where C=100 μF.

FIG. 4 is a graph illustrating the acoustic signals produced by the system of FIG. 1 in a case where, during calibration, the ultrasound waves were interacting with a polymethylmethacrylate (“PMMA”) film.

FIG. 5 is a graph illustrating the displacement of the PMMA film during the operation the system of FIG. 10.

FIG. 6 is a graph illustrating the surface and recorded pressure impulses at different lasers voltages for the system of FIG. 1.

FIG. 7 shows the ultrasound pressure profile of the system of FIG. 1 as a function of distance from the source.

FIG. 8 is a converter for converting optical energy from a laser to ultrasonic energy.

FIG. 9 is a graph illustrating the acoustic wave amplitudes as a function of time, which were generated using different optical energy absorbing media in the converter of FIG. 8.

FIG. 10 is a graph illustrating the acoustic wave amplitudes as a function of input energy from a laser, which were generated using different optical energy absorbing media in the converter of FIG. 8.

FIG. 11 is a graph that illustrates the acoustic pressures as a function of increasing laser energy generated by the optical-acoustic converter of FIG. 8 where the absorption medium included a solid acrylic dye.

FIG. 12 is a graph that illustrates the amplitude of an ultrasonic output signal of the converter of FIG. 8 as a function of time.

FIG. 13 is a graph that illustrates a fast Fourier transform (“FFT”) analysis of the output signal of FIG. 12.

FIG. 14 is a graph that illustrates the amplitude of an ultrasonic output signal of an alternate embodiment of the converter of FIG. 8 as a function of time.

FIG. 15 is a graph that illustrates an FFT analysis of the output signal of FIG. 14.

FIG. 16 is a graph illustrating an axial profile of an acoustic wave generated by the optical-acoustic converter of FIG. 8.

FIGS. 17-18 are graphs illustrating exemplary radial profiles for acoustic pressure waves generated with the converter of FIG. 8.

FIG. 19 is a graph illustrating an axial profile of an acoustic wave generated by an alternate embodiment of the optical-acoustic converter of FIG. 8.

FIGS. 20-21 are graphs illustrating exemplary radial profiles of acoustic pressure waves generated by an alternate embodiment of the optical-acoustic converter of FIG. 8.

FIG. 22 is a graph illustrating the pressure at the outputs of the converter of FIG. 8 and of an alternate embodiment of that converter.

FIG. 23 is a graph illustrating three test cases of an optical-acoustic converter used on swine fat.

FIG. 24 is an alternate embodiment of a converter including a piezoelement.

FIG. 25 is another alternate embodiment of a converter including a piezoelement.

FIG. 26 is a set of graphs illustrating typical output parameters measured using embodiments similar to the converters of FIGS. 24 and 25, including the dependences of the voltage on the transducer, the displacement of a PMMA mirror surface at the focal region of the transducer, and the speed of the displacement.

FIG. 27 is a set of graphs illustrating exemplary resonant frequencies for hair having various lengths.

### DETAILED DESCRIPTION

Ultrasonic energy can be delivered to hard and soft tissue using laser-based techniques. For example, sharply focused high-intensity high-frequency beams of ultrasound can be generated using a short-pulse laser. Examples of laser-based techniques for generation of high-frequency ultrasound are described in several sources, including Scruby, C. B., and Drain, L. E., Laser Ultrasonics: Techniques and Applications. Adam-Hilger, New York (1990)). Generally, the primary source in the generation of ultrasound waves is thermal expansion of the illuminated laser material. The release of thermal energy in the media is much smaller than the heat of vaporization and no phase transition is involved.

Many mechanisms and processes are involved in generating ultrasonic energy, such as evaporation of the media and phase transitions at higher densities of the laser energy. Although the process of generating ultrasonic waves is complicated, the amplitude of the generated ultrasound wave can be estimated from the following equation:

$${P}_{0} ≅ \frac{1}{2 {C}_{P}} {E}_{S} ( 1 - R ) {α}_{L} {C}_{0}^{2} {β}_{T}$$

where E_{S}(1−R) is the density of the absorbed energy, α_{L} is the absorption coefficient, Co is the speed of sound, β_{T} is the thermal expansion constant, and C_{P} is the thermal capacity of the material.

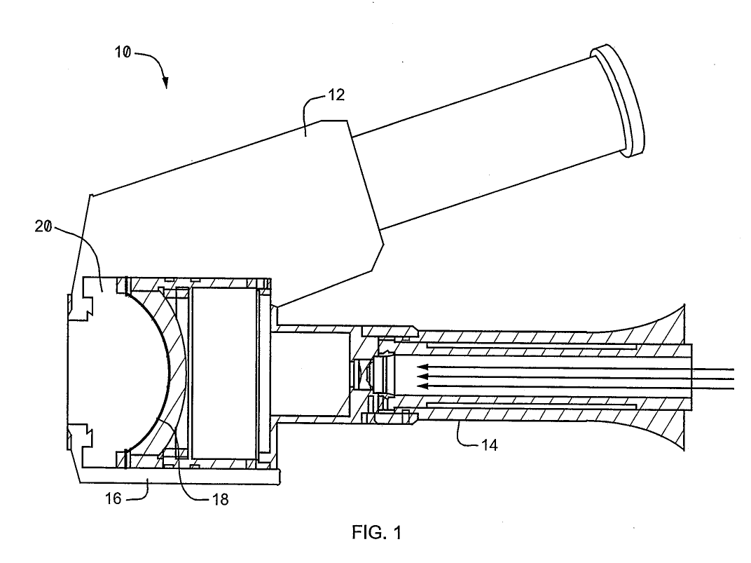

To generate the optimal amplitude of the ultrasound signal for use in treating tissue, a short laser impulse can be used with a media having large absorbance, large volume expansion coefficient, and low thermal capacity. Referring to FIGS. 1-3, one exemplary embodiment is a laser-based ultrasonic system 10, which generates ultrasonic stress waves using a laser. System 10 includes a reservoir 12, a laser 14, a coupling chamber 16 and a focusing member 18. Reservoir 12 contains a medium that is supplied to chamber 16 via an opening 20, which can include a valve or other suitable arrangement. In system 10, the medium is supplied by the application of positive pressure to reservoir 12, but many other configurations are possible including a gravity feed or other mechanism. In still other embodiments, an additional reservoir of medium may not be included. The medium is system 10 is a gel. In other embodiments, other media could be used such as a water supply or other suitable substance, mixture, composition, etc. Laser 14 is a short-pulse Nd:Yag. Focusing member 18 can be constructed many different ways, including as a single member or a combination of suitable parts and or elements. In system 10, focusing member 18 is a steel shield with a laser target and focusing system located internally.

System 10 generates ultrasound by illuminating focusing member 18 with laser 14. The laser energy absorbed by the material of focusing member 18 causes localized heating with accompanying thermal expansion. Absorption of the incident pulse energy and the associated temperature gradients induce a rapidly changing strain field. The strain field, in turn, radiates energy as elastic (ultrasonic) waves. A traveling acoustic wave thereby propagates through the member 18 and can be focused on the targeted tissue through the coupling chamber 16. The traveling acoustic wave propagates through the medium contained in coupling chamber 16, which is supplied from reservoir 12.

Preferably, system 10 is calibrated. Calibration can be accomplished using a hydrophone or Michelson interferometer. For example, ultrasound can be detected by measuring the displacement of the thin polymethylmethacrylate (“PMMA”) mirror film under the ultrasound action. In one experimental set up, one of the interferometer's laser beams was sent to the PMMA mirror and the other beam was sent to a reference mirror. Upon reflection, the two beams were recombined parallel to each other and made to interfere at the photodetector. The minimum displacement sensitivity of the interferometer used that experiment was approximately 10⁻¹⁰ m.

When measuring the mirror's vibration caused by ultrasound, the output of the interferometer was proportional to the ultrasonic displacement:

$$P = ρ c \frac{dS}{dt}$$

where P is the pressure developed in the PMMA film; ρ is the density of PMMA; c is the speed of sound (in PMMA); and dS/dt is the speed of the displacement of the PMMA surface. The displacement S can be determined by the equation:

$$S = \frac{λ}{4} \frac{{V}_{exp}}{{V}_{int}}$$

where Vₑₓₚ and Vᵢₙₜ are the amplitudes recorded from the photodetector during a measurement and at the condition when shift of the interferometer's bands is more than λ/4.

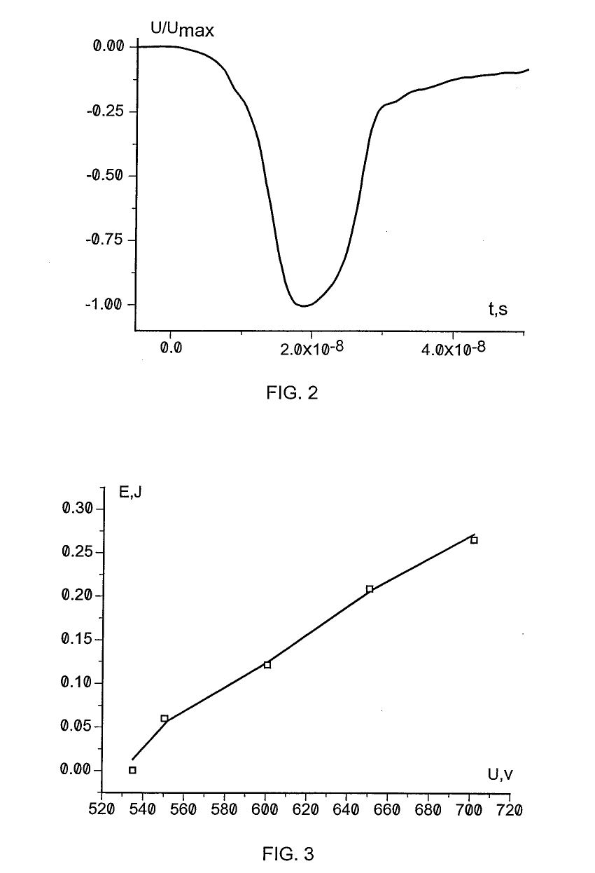

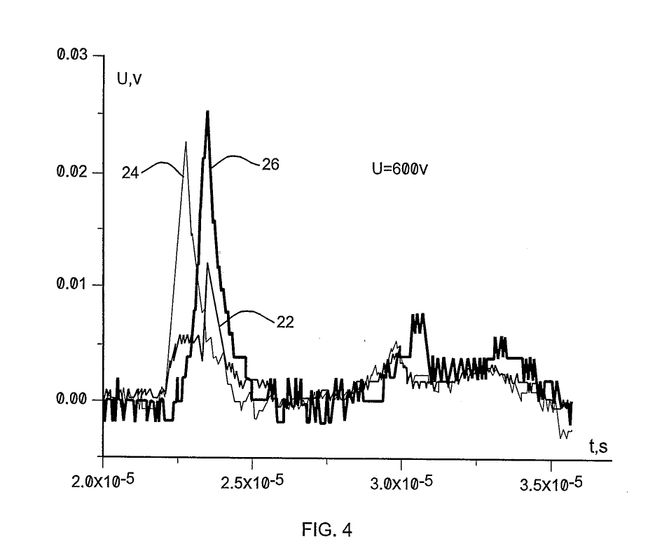

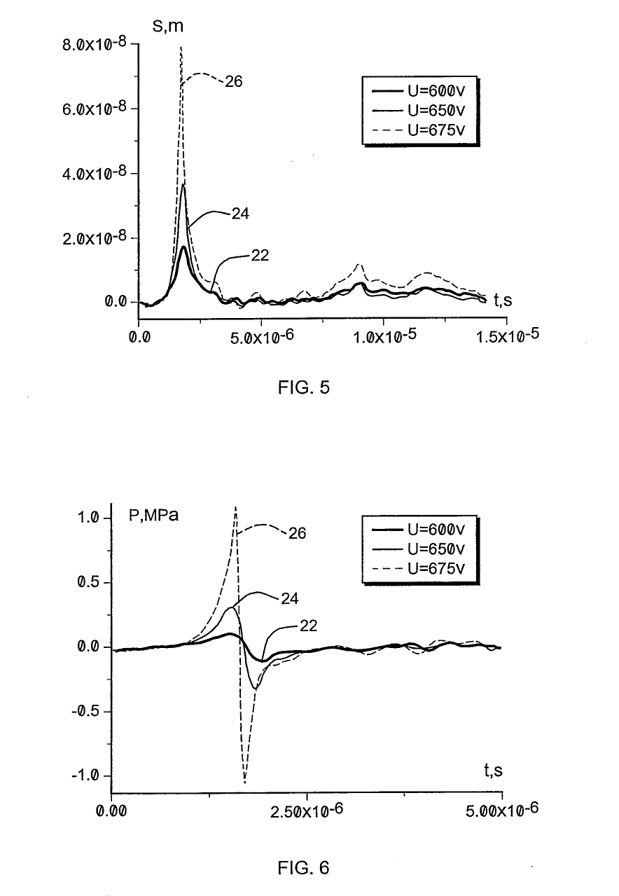

Referring to FIG. 2, system 10 produced laser impulse as shown during calibration. The laser impulses are shown as a ratio of the laser impulse to the maximum laser impulse. Referring to FIG. 3, system 10 produced ultrasound having an output energy that increased as a function of pumping voltage as shown. Referring to FIGS. 4-6, the parameters of three exemplary output ultrasound signals are shown. Output signals 22, 24, and 26 were produced when the pumping voltages respectively were 600 V, 650 V, and 675 V. Referring to FIG. 4, the acoustic signals produced by the system of FIG. 1 are shown in a case where, during calibration, the ultrasound waves were interacting with a PMMA film. Similarly, FIG. 5 illustrates the displacement of the PMMA film during the operation the system 10. Referring to FIG. 6, the recorded pressure impulses at different laser voltages are shown.

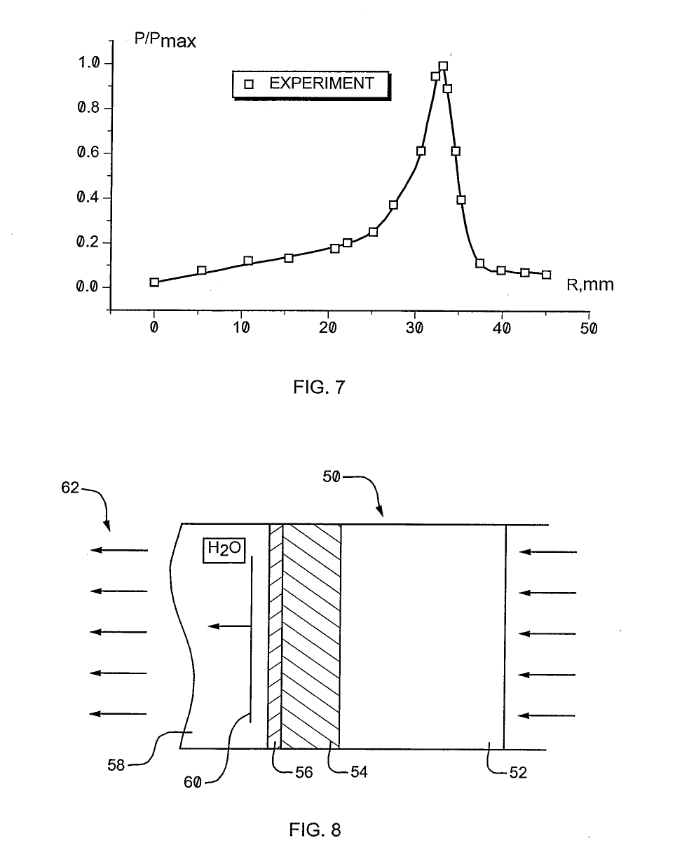

Referring to FIG. 7, the relative ultrasound pressures confirms that the ultrasound waves produced by system 10 were focused. FIG. 7 shows the ultrasound pressure profile as a function of distance from the source.

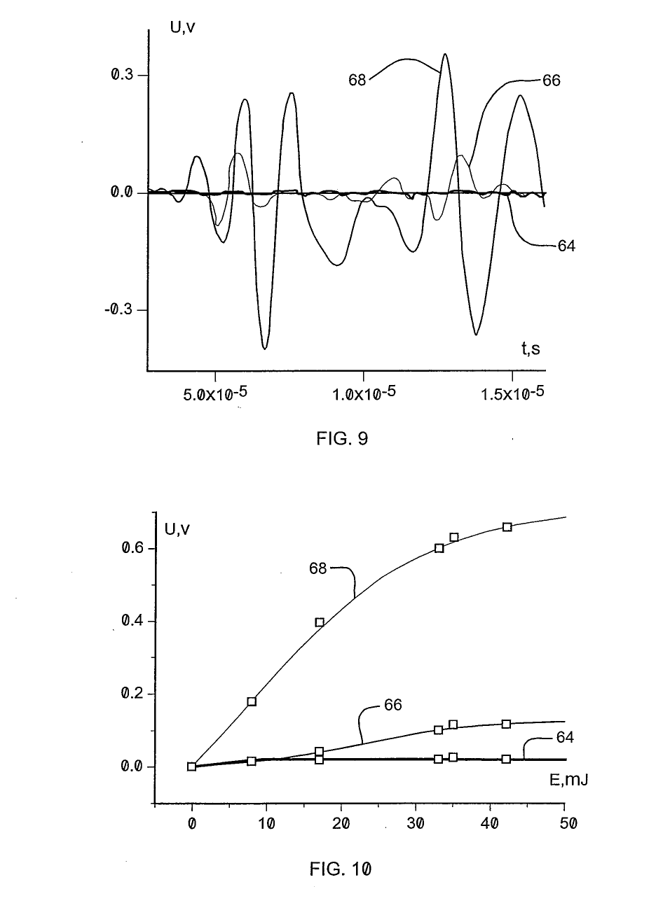

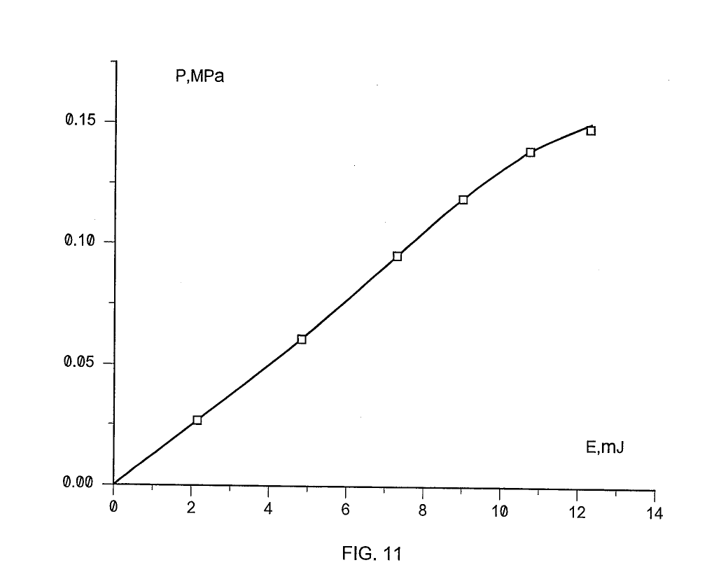

In other embodiments, the laser-generated ultrasound can be generated using different configurations and different media. For example, an optical-to-acoustic converter 50 is shown in FIG. 8. Converter 50 includes an optical element 52, and an absorbing medium 54, a translation member 56 and an output medium 58. Lens 52 is a PMMA lens that is neither convergent nor divergent (although many other configurations are possible). Absorbing medium 54 is 1 mm thick. Translation member 56 is a polymer having a thickness of 0.3 mm. Output medium 58 is a chamber 60 filled with water. The output acoustic waves 62 vary depending on the absorbing medium that is used. For example, referring to FIGS. 9 and 10, the output profiles of the acoustic waves as a function of time are shown for three cases. Output wave 64 corresponds to an absorbing medium of In—Ga eutectics; Output wave 66 corresponds to an absorbing medium of birch tar; and output wave 68 corresponds to an absorbing medium of birch tar mixed with acrylic dye. In the later case, a composition of 25-30% acrylic dye is thought to be preferred. FIG. 11 demonstrates dependence of the generated pressure in the solid acrylic layer with increasing laser energy where a solid acrylic dye was used. At the same laser energy exposures, inorganic components demonstrated the worst efficiency in transformation of the laser energy into the acoustic energy.

The phenomena observed during experiments conducted using converter 50 are thought to be caused by the difference between the acoustic impedance of water and the impedance of In—Ga eutectics used (which is one order of magnitude higher than for water). Acoustic impedances of the organic media used in the experiments were close to that of water. Additionally, nonlinear relations between the generated acoustic pressure and the laser energy p=f(E)) could be detected at energies (E) of approximately 10-15 mJ in the case of In—Ga eutectics, while the relationship was is 2-3 times greater for the organic absorption media.

The optical absorbance of the eutectics is large, and local spots providing an optical shortcut could result. It is possible that cavitations in such spots would suppress the intensity of the generated acoustic waves, which would make a converter using such absorption material less efficient. Organic liquids are presently considered preferable for use as an absorbing media, for the reasons discussed above and also because the Gruneisen's coefficients of such media provide a profound thermoelastic effect, which is not required but is preferred.

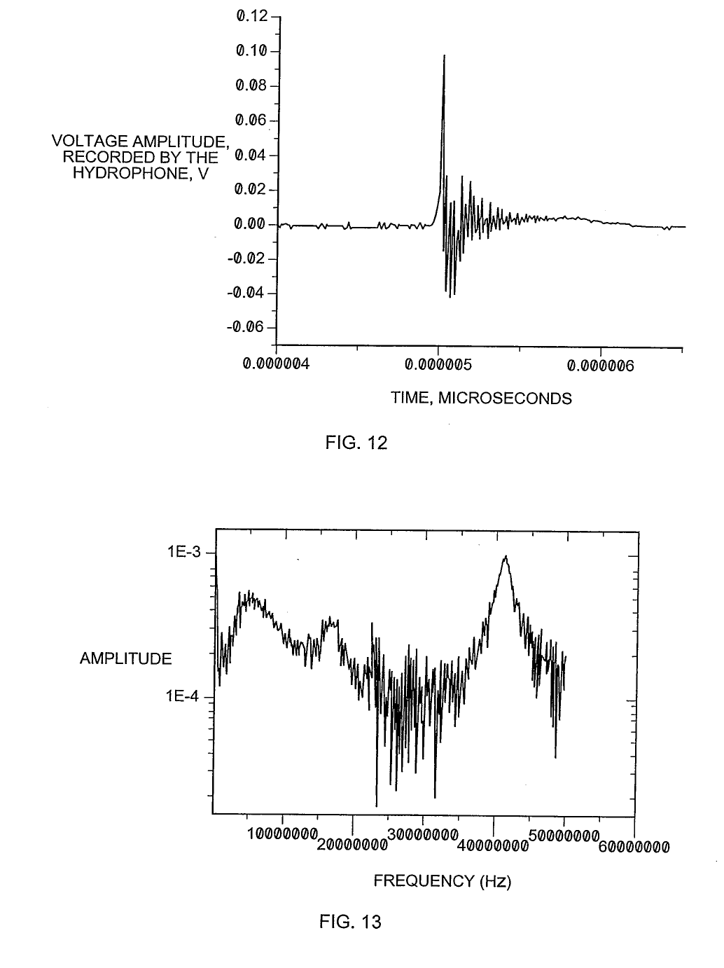

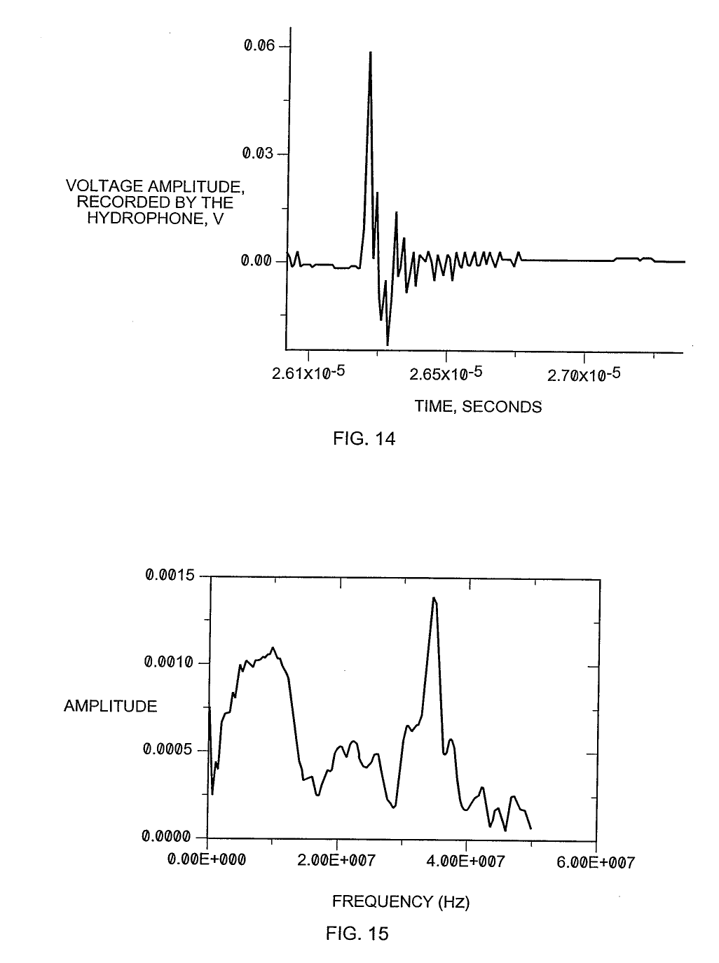

In an additional experiment, converter 50 was modified to instead provide an optical-acoustic converter having a solid dried acrylic layer. To measure the geometry of the generated ultrasound waves, a wide-band high sensitive commercial hydrophone (HGL-0200, Onda corp.) and a 3-D positioning system (Velmex inc, with a 6 μm step on each slide) were used. The measured laser energy was used in the range of 0.1-0.45 J. Laser-induced acoustic signals were generated using both converters: one having an absorption medium of birch tar and the other further including the solid acrylic dye. Referring to FIGS. 12-15, a fast Fourier transform (“FFT”) analysis of the recorded signals revealed the presence of a broad band of approximately 40-50 MHz. FIGS. 12 and 13 illustrate the case where an input laser energy of 0.15 J was applied to the converter having the liquid birch tar. FIG. 12 illustrates the amplitude of the output signal as a function of time, and FIG. 13 illustrates the FFT analysis of the output signal. Similarly, FIGS. 14 and 15 illustrate the case where an input laser energy of 0.21 J was applied to the converter having the solid acrylic layer. FIG. 14 illustrates the amplitude of the output signal as a function of time, and FIG. 15 illustrates the FFT analysis of the output signal.

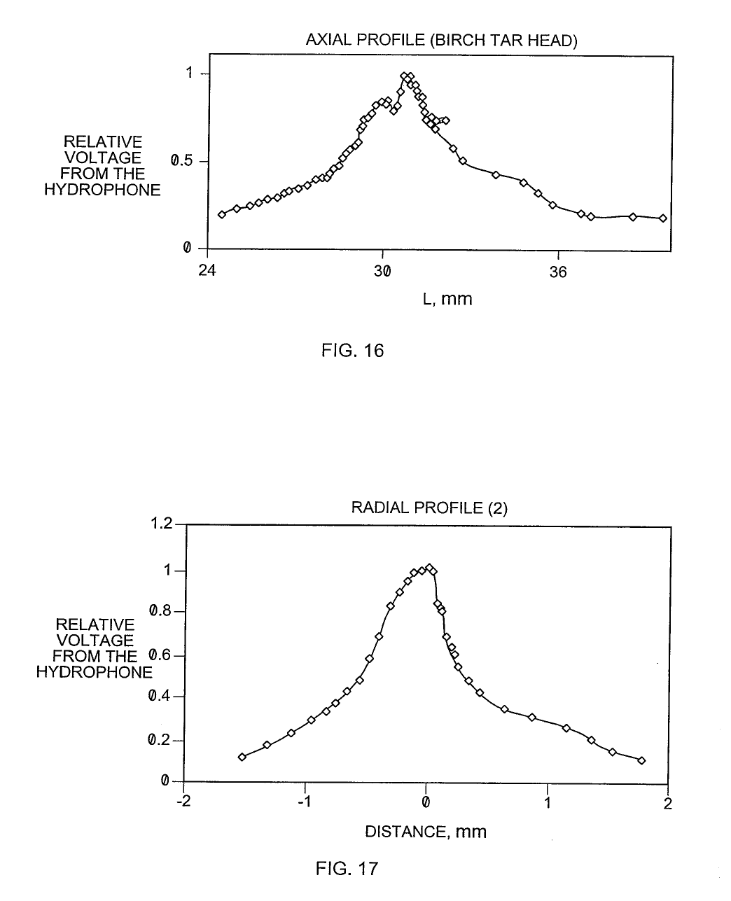

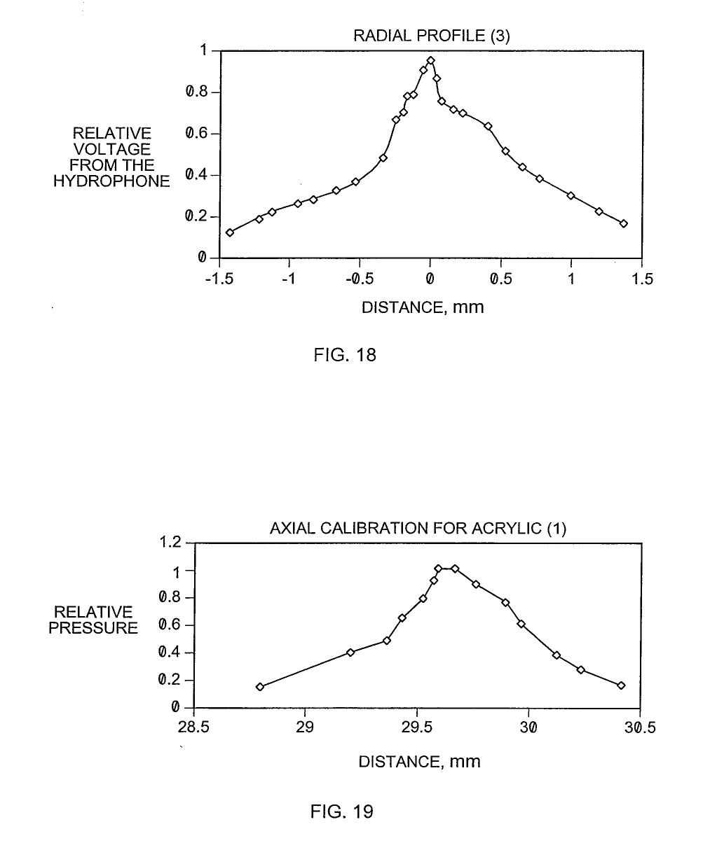

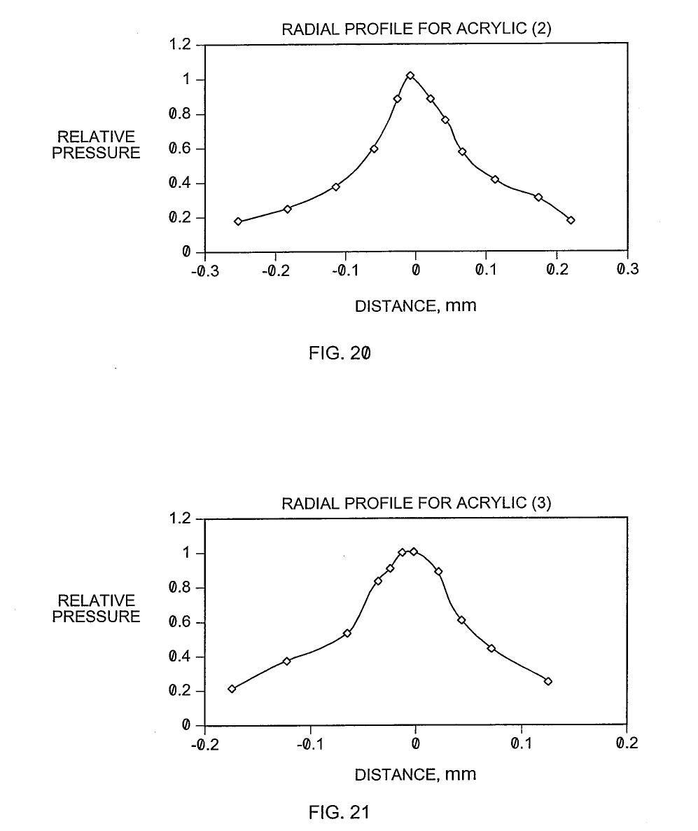

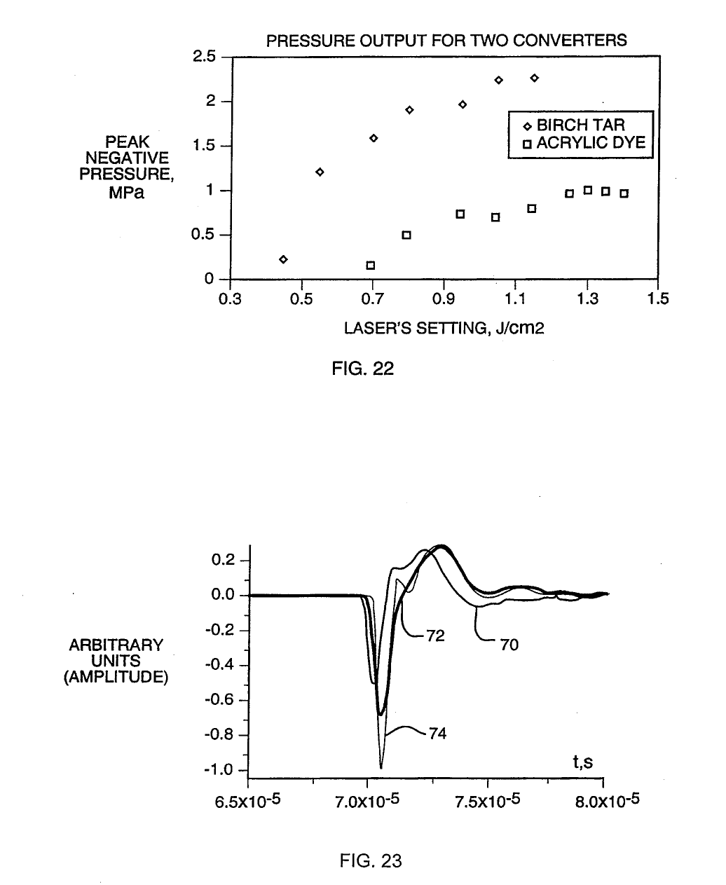

Referring to FIGS. 16-21, a 3-D position system with a 6 μm step among each slide was used to obtain acoustic profiles generated by both ultrasonic converters analyzed in conjunction with FIGS. 12-15. FIG. 16 shows an axial profile for the converter filled with birch tar shown, while two radial measurements of that embodiment are shown in FIGS. 17 and 18. Similarly, FIG. 19 shows an axial profile for the converter having the solid acrylic, while two radial measurements of that embodiment are shown in FIGS. 20 and 21.

Based on the data obtained from testing these two embodiments, the focal region for each optical-acoustic converter can be determined. Each converter provides sharp focused acoustic regions.

Referring to FIG. 21, pressure measurements for the output waves of each converter are shown. The pressure measurements were obtained using a hydrophone that was located within the focal region of each output. The voltage output from the hydrophone was detected at different laser energies. The pressure measurement were then used to calculate the acoustic pressures taking into account the known sensitivity of the hydrophone, which is conveniently flat along a very wide frequency band (1-20 MHz). Over the same energy ranges, the converter filled with the liquid birch tar generated a higher acoustic pressure than the alternate embodiment having the solid acrylic dye at the same energies of the laser. However, the use of the liquid birch tar is not required, and converters having many different configurations are possible.

Optical-acoustic energy converters and systems, such as those described above, can be use for the treatment of fatty tissue, especially in the human body. By applying a focused acoustic beam beneath the skin, targeted adipose tissue can be broken down by the high intensity energy. There are several mechanisms that are thought to affect the tissue being irradiated with the acoustic waves. For example, depending on acoustic frequency, ultrasound intensity, and viscosity of the medium, the acoustic wave can cause a rise in temperature that is secondary to the direct absorption of ultrasonic energy. Additional mechanical processes such as streaming, shear stressing, and cavitation can play a role when relatively higher acoustic pressures are used.

In one experiment using a converter with a solid acrylic dye layer, the application of 250 acoustic impulses at an energy of 250 mJ destroyed swine fat at a depth of 3 mm under the skin layer and having an area of approximately 0.25 mm². Referring to FIG. 23, changes in the levels of ultrasound pressure when an ultrasound signal passed through the swine fat are illustrated. Pressure curve 70 represents the case where the fat had a thickness of 3 mm and contained muscle inclusions. Pressure curve 72 represents the case where the fat had a thickness of 3 mm and contained no muscle inclusions. Pressure curve 74 represents the case where output of the converter was measured through water (10 mm) instead of swine fat. The estimated absorbance coefficient from the measurements illustrated in FIG. 23 are K_{f}=1.4 cm⁻¹ for swine fat without muscles and K_{fm}=2.5 cm⁻¹ in case when fat containing some muscles inclusions.

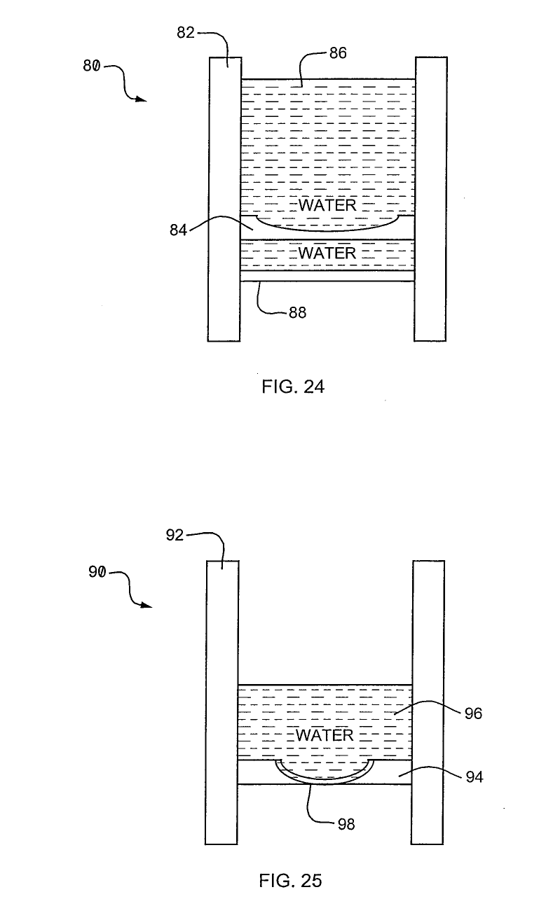

In another embodiment, a method to generate and focus ultrasound energy includes using piezoelectric elements, e.g., spherically-shaped elements. Referring to FIG. 24, a converter 80 includes housing 82, a polymer lens 84 contained in housing 82 and surrounded by an optical medium 86 (water in this embodiment), and a piezoelement 88. In an alternate configuration, a converter 90 includes a housing 92 containing a focusing support member 94, an optical medium 96 (water in this embodiment), and a piezoelement 98. In both embodiments, the curved and focused piezoelements 88 and 98 were shielded and assembled into ultrasound transducers. The ultrasound transducers had a resonant frequency of 6 MHz, but many other configurations are possible.

In converter 80, optical energy is passed through lens 84 and focused onto piezoelement 88. In contrast, the optical energy in converter 90 is focused by the piezoelement 98 itself, and not a lens. In that particular embodiment, the support member 94 fixes the piezoelement 98 in the desired configuration. However, many other embodiments, including many additional configurations for converters similar to converters 80 and 90, are possible. Exemplary specifications for the piezoelements 88 and 98 are provided in Table 1.

TABLE 1
Characteristics of Exemplary Piezoceramic Material CTS-191.

| | |
|---|---|
| Thickness, mm | 0.4 |
| Diameters, mm | 40 (piezoelement 88) |
| | 22 (piezoelement 98) |
| Geometrical radius, mm | 35 |
| Estimated focal radius, mm | 15 |
| ε¹/ε₀ | 960-1000 |
| k_{ρ} | 0.52-0.55 |
| d₃₁, Kl/N | (98-100)*10⁻¹² |
| g₃₁, V · m/N | (12-13)*10⁻³ |
| Q | 200-220 |
| tg δ, (at 1 kHz) | 0.006-0.009 |
| Tₖ, ° C. | 360 |
| Resonance frequency of the | 5.9 ± 0.05 |
| transducers, MHz | |

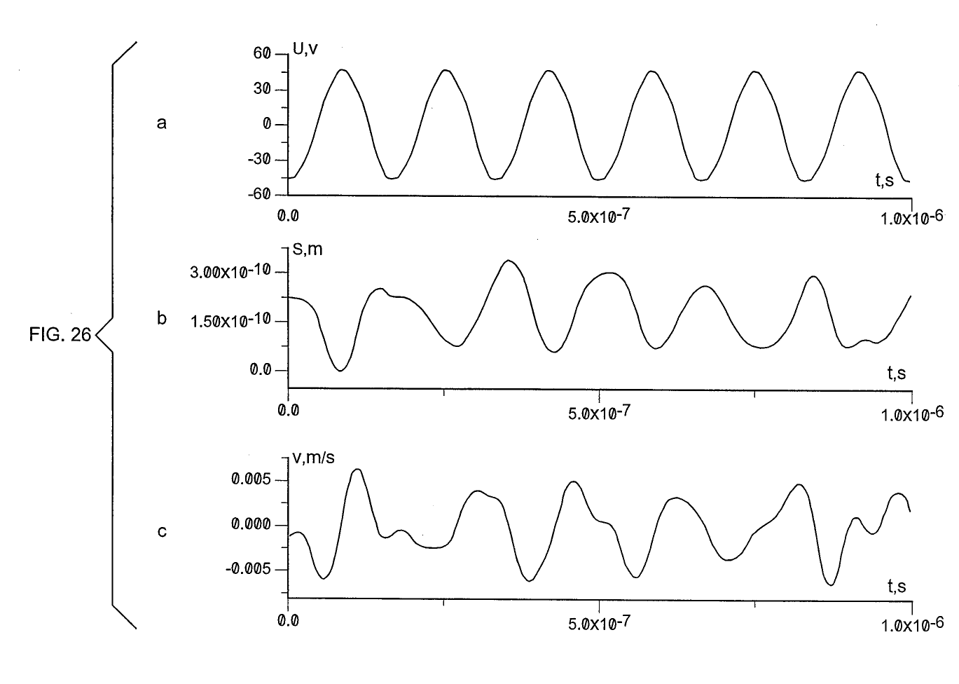

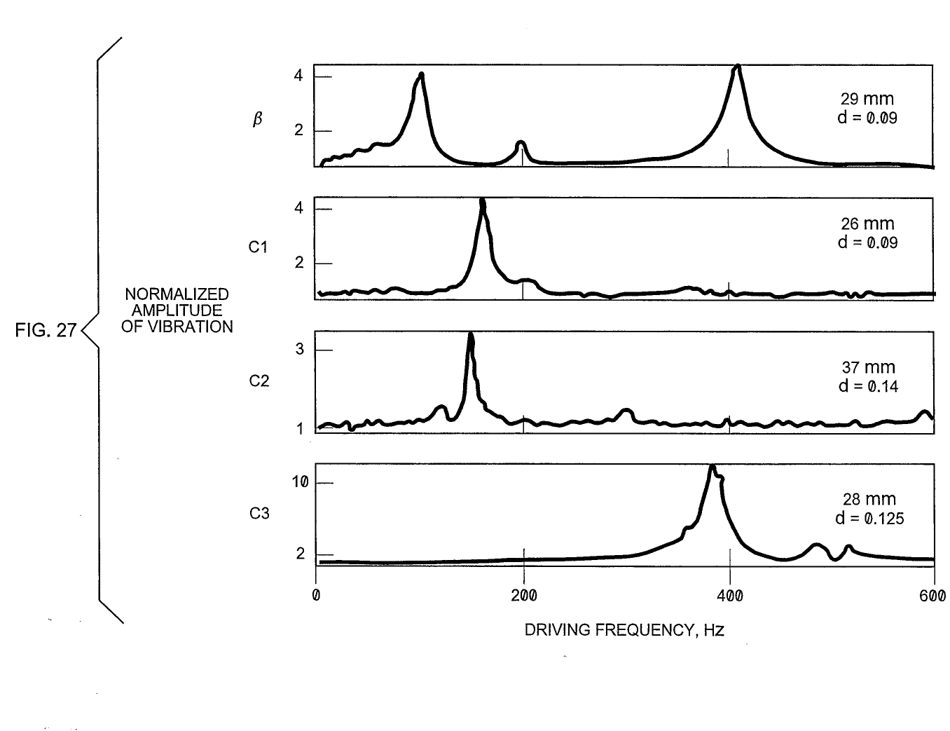

In order to measure the acoustic pressures generated with piezoceramic transducers, the same method employing Michelson interferometer as for optical-acoustics described above was used. FIG. 26 illustrates the changes in voltage on the transducers (a), measured displacement of the PMMA surface placed at the ultrasound focal region (b), and calculated displacement's speed (c). Evaluation of the pressure amplitude on the water-PMMA interface showed pressures around 0.02 MPa. As a side effect, degradation of the PMMA surface under ultrasound's action was observed. Deformation of the PMMA mirror was attributed to the thermal elevation at the focal region of continuous 6 MHz ultrasound used due to its absorption by PMMA (Tg=90° C.).

Samples of swine fat having a thickness of 40 mm were insonated with a piezoceramic device using continuous 6 MHz ultrasound generated by the manufactured transducers. The fat included areas of destruction at the focal region of the transducers after samples were insonated for 10 min.

In still another embodiment employing piezoelements as part of the transducer, an array of piezoelements can be used. Another embodiment of the transducers for fat destruction are shown on the FIG. 38. It is the array type transducer. This allows several piezoceramic or other piezoelectric elements to be combined into an array to cover larger insonation areas and/or to concentrate more ultrasound energy for treatment.

There are many potential uses for focused or concentrated ultrasound using optical-acoustic converters or other types of transducers. Several examples are discussed below.

### Selective Sonothermolysis

Such devices can be used to create controlled zones of hyperthermia and thermal damage in tissue (selective sonothermolysis). A principle similar to selective photothermolysis in photomedicine can be formulated for the ultrasound applications. Specifically, localized and controlled zone of hyperthermia and/or thermal damage can be created when:
1. Absorption coefficient of ultrasound in the targeted area is higher than in surrounding tissue; and
2. Duration of the ultrasonic pulse tₛ is shorter than the thermal relaxation time of the targeted area.
   Also by analogy with photomedicine, extended form of this principle can be formulated that will encompass situation when chromophore is physically separated from the targeted area.

The above-formulated principle of selective sonothermolysis can be utilized, for example, for targeting protein-rich structures embedded into tissues with lower protein content. One example of such a configuration is fibrous septa in subcutaneous tissue. Tissues reach in proteins (such as septa—connective tissue) typically demonstrate higher absorption of ultrasound than protein-poor tissues such as subcutaneous fat.

### Treatment of Cellulite

Most of the current noninvasive methods to treat cellulite, such as ingested capsules, massage combined with heat or laser treatment, etc., usually have side effects and have little to no effect or, if any effect, only a temporary effect. Embodiments of the invention can be used to focus ultrasound energy to modify the tissue structure and reduce or eliminate cellulite. The cell debris and released content will be absorbed by macrophage cells and naturally eliminated by the organism. Tissue in the treated area resorbs over time, resulting in reduced volume. Some embodiments are based on the principle of selective photothermolysis described above. The fibrous septa in the fat are thermally modified by the ultrasonic energy in order to reduce tension to the skin.

Improvement of Skin Appearance, Tone, and/or Texture

The principle of selective sonothermolysis can be used to heat denser, elastin-rich areas in the skin, stimulating new collagen production and shrinkage of the dermal interstitial matrix.

### Tattoo Removal by Ultrasound-Assisted Dye Diffusion

Dyes and inks can be forced deeper into tissue where they cannot be seen and where the body can be able to remove them. Due to the optical properties of skin tissue, pigment cannot be seen by the human eye below a few hundred microns in depth into the tissue. Experiments demonstrate that ultrasound applied to tissue containing tattoo pigment or other similar particles may be forced deep into the tissue and therefore make it less visible.

In one experiment, tissue containing a dye on the surface was treated with ultrasound using 75 kHz and 118 kHz sonicators from Titan. The upper layer of the treated tissue was stained with a dye and then left for several days to monitor the diffusion of the dye into the tissue. It was found that the used dye had a very low diffusivity in the fat tissue and no penetration was detected.

However, ultrasound action on the dye was apparent. Histology demonstrated that traces of the dye were observed in the fatty tissue. Samples of the fatty tissue were cut 20 minutes following treatment with low frequency ultrasound on a skin surface stained with a blue dye. The dye penetrated 1.5-2 mm into the tissue from the surface of the tissue. Diffusion rate of the blue dye into the fatty tissue depended on the duration of ultrasound insonation.

### Hair Removal and Reduction

For hair removal with aid of ultrasound, a different approach is used than has been used previously. It is not efficient in practice to attempt to focus the ultrasound beam on individual hair roots. Such a technique would be time consuming, and potentially ineffective. Instead, preferably ultrasound is applied to cause a resonance absorbance by the hair complex. Ultrasound of a resonant frequency can induce damage to arrest hair growth.

Several authors have reported recently that they observed a resonance behavior in the rat vibrissae when it was driven with a piezoelectric stimulator. (See Andermann M. L., et al. Neuron, V.42, 451-463 (2004); and Neimark M. A., et al. J. Neurosci. V.23, 6499-6509 (2003), which are incorporated by reference.) Those authors also noted also that longer vibrissae displayed lower resonance frequencies that could be important observation for the practical implementation of the ultrasound energy (see the pictures below).

In some embodiments, tissue is insonated using a wide skin area. The ultrasound frequency is selected to cause a selective resonance absorption of the ultrasound energy by the hair. Examples of suitable resonance frequencies for various hair lengths are shown in FIG.

Some embodiments use ultrasonic/acoustical energy tuned to one of a resonant frequency of a hair shaft, the inner root sheath of the follicle, outer root sheath of the follicle, and the hair matrix. Operation at the resonant frequency for a period of time affects the mechanical interface between these structures, e.g., for a period of a few seconds or shorter depending on the treatment parameters. If sufficient ultrasonic energy is applied, normal mechanism of hair growth, i.e. creeping movement of inner root sheath (IRS) with respect to outer root sheath (ORS) can be compromised or completely disrupted, thus substantially slowing down or completely arresting hair growth. Since a range of hair lengths and diameters is present in the skin, the ultrasound frequency needs to be varied, either by sweeping or by using pulsed sources.

The vibration mechanism is based partially on the water content of the structures involved. The water content affects acoustic properties of the inner root sheath and the hair shaft versus the outer root sheath. The structures have the following approximate water content: Inner root sheath, Hair Shaft (predominant constituent—Keratin) 15-30% H₂O; Outer root sheath, Dermis 75% H₂O.

The resonant frequencies for the vibration modes of these structures can be approximated by the following equation:

Ωᵣ=vₛ/d;

where Ωᵣ is the resonant frequency; vₛ is the speed of sound; and d is the length of hair shaft. Other hair shaft dimensions can also be used to approximate the resonance frequency.

The hair shaft, inner sheath, outer sheath will oscillate in various directions as a function of the fundamental frequency and harmonics generated by the ultrasound device. Ultrasonic waves in solid bodies such as hair structures can be longitudinal, transversal, torsion or bending. Sound velocity and, therefore, resonant frequency are, strictly speaking, dependent on the type of wave. As a result, different frequencies can excite resonances of different types.

In operation the frequency of the acoustic energy is adjusted to one or more of the resonant frequencies of the hair shaft, inner sheath, outer sheath to induce vibrations which will cause mechanical disconnection of the structures coupling the hair shaft inner sheath, outer sheath and damaging the mechanism responsible for lifting the hair shaft.

In one embodiment, the frequency of ultrasound is swept over the range of interest. In another embodiment, an ultrasonic pulse is applied, which contains a broad range of frequencies. The distal area of the device can include a plurality of transducers sharing a single frequency/pulse generator through an energy distribution network and activated in sequence. This approach can reduce cost of the device. In preferred embodiments, there is a coupling medium providing acoustical contact between the transducer(s) and hairs. This medium can be a gel, a liquid, a film, or some other implement. Physical properties of the coupling medium should be selected in such a way as to favor coupling of ultrasonic energy into hairs and not into skin. This can be achieved due to differences in the velocities of sound (about 1700 m/s for hair and about 1500 m/s for skin). Exemplary parameters are shown in Table II.

TABLE II
Exemplary Treatment Parameters for Hair Removal

| | |
|---|---|
| Frequency | 20 kHz to 50 MHz |
| power density | 0.1 W/cm² to 10 W/cm² |
| exposure time | 0.1 s to 10 min |

This process advantageously requires less energy than analogous optical, thermal, or mechanical procedures. This device damages and/or destroys the mechanical connections among hair shaft inner root sheath, outer root sheath when the elastic limits of these structures are exceeded. As a consequence of the damage, the hair lifting mechanism is damaged or destroyed thereby reducing hair growth rates in the treatment area. In some embodiments, the ultrasonic member can be combined with a mechanical depilatory member to pull the hairs with damaged or disrupted IRS/ORS interface out. The members can be combined in a device, which is scanned across skin surface.

In another embodiment, an ultrasonic device for hair removal and/or permanent hair reduction is provided in a handheld device suitable for use by a consumer. Given the nature of the ultrasonic energy, such a device could be relatively safe and effective as a handheld device, and it would not present some of the safety concerns encountered with certain wavelengths of electromagnetic radiation such as eye safety or other safety issues unique to optical radiation.

In another embodiment, the resonant ultrasonic energy can affect the nerve endings in the skin and reduce pain caused by depilation. Use of vibration to mitigate pain is known in the art, but ultrasonic energy for that purpose can be more effective.

In some embodiments, electrostatic or mechanical preparation can be conducted to provide optimal positioning of the hairs prior to application of ultrasonic energy. This process and device can be useful in cosmetic applications (e.g. delaying beard growth) and also can be used to treat PFB or reduce the need for shaving. For example, hair grows at a rate about 100-200 μm per day. By reducing the lifting of the hair shaft to 100 μm per day a substantial improvement in reducing, for example, shaving-related problems or PFB problem can be achieved. By employing acoustical waveguides (e.g. horns) or other focusing devices more mechanical damage can be obtained. In addition, at higher energy levels, heating of the tissue can also provide increased damage to the hair lifting mechanism.

The device can also include a detector to determine when the energy is at a resonant frequency and is inducing vibration in one of the structures to be damaged. The detector can be, for example, a microphone or vibration sensor. The detector can be used to provide feedback to control the acoustic frequency or the energy level of the signal. When the acoustic energy is close to a resonant frequency, the mechanical oscillation and vibration of the hair shaft can be observed.

A similar concept can be used for selective heating of a hair follicle and hair removal due to thermal acoustic effects. At resonant frequencies of the hair shaft or/and inner root sheath acoustic energy can be deposited as heat in the hair follicle. This acoustic heating can be combined with selective light heating through melanin absorption. Another advantage is that the device can apply a large beam scan because focusing is not required in contrast to applications which use focused acoustic energy to cause thermal damage.

Resonant frequencies can be estimated based on the hair shaft/inner root sheath using the following equations. The speed of SOS in hair is expected to be larger than in the epidermis (1642 cm/s) but much smaller than in bone (3375 m/s). Thus, in the following cases, the following parameters were used: c=2000 and m/s=2·10⁶ mm/s. Because the hair shaft and inner root sheath are essentially a close cylindrical cavity of finite length L, the Eigen frequencies are given by relation (1):

$$ν = \frac{c}{2 \cdot π} \cdot \sqrt{{k}_{l}^{2} + \frac{{n}^{2} \cdot {π}^{2}}{{L}^{2}}} , \qquad ( 1 )$$

The ultrasonic wave equation is thought to be formulated in terms of dilatation (the dilatation is the dependent variable). The following are exemplary cases of longitudinal hair oscillations and transverse hair oscillations. The transverse wave number (the first term in (1)) is set to be 0, therefore:

$$v = \frac{c \cdot n}{2 \cdot L} \qquad ( 2 )$$

For n=1 one gets:

| L, mm | ν_{Long}, MHz |
|---|---|
| 1 | 1 |
| 3 | 0.333 |
| 5 | 0.2 |

For transverse hair oscillations:

$$For n = 0 yielding \text{:} v = \frac{c}{2 \cdot π} \cdot {k}_{l} = \frac{c}{2 \cdot π} \cdot \frac{{j}_{l}}{a} , \qquad ( 3 )$$

where j₁ is the zero of the appropriate Bessel function (depending on the mode), a is the hair radius. Set 1=0 to get the lowest Eigen frequency. If the side surface is clamped, then j₀≈2.4 yielding:

| 2 · α (hair diameter), μm | ν_{Trans}, MHz |
|---|---|
| 50 | 31 |
| 100 | 15 |
| 150 | 10 |

If the side surface is free, then j₀≈1.84 yielding:

| 2 · α (hair diameter), μm | ν_{Trans}, MHz |
|---|---|
| 50 | 24 |
| 100 | 12 |
| 150 | 7.7 |

The actual Eigen frequencies should be in between these limits. So a range of resonance frequency of 31 MHZ is expected.

Ultrasound can cause elevation of temperature in the medium due to its absorption. In order to localize and to visualize the profile of the focal region, it is convenient to employ a transparent gel which will become opaque when heated. The following protocol is to prepare such thermo-sensitive gel based on the polyacrylamide: dissolve 1 egg's white in 50 mL of pure distilled water; add 8.2 g of acrylamide and 0.42 g of bis-acrylamide (N,N′-methylene-bis-acrylamide); in a separate 10 mL of water dissolve 30 μL TEMED (tetramethylethylenediamide) and 0.06 g of ammonium persulfate; add 10 mL of the last prepared solution to the 50 mL previously prepared solution, mixing well and avoiding bubbles. Pour the mixture into a beaker and leave for 2-3 h at room temperature to complete polymerization. The resulting gel is transparent but it becomes opaque at temperature around 60° C. This gel can be stored in a tightly closed baker to avoid air exposure and drying the gel.

The patent, scientific and medical publications referred to herein establish knowledge that was available to those of ordinary skill in the art at the time the invention was made. The entire disclosures of the issued U.S. patents, published and pending patent applications, and other references cited herein are hereby incorporated by reference in their entirety.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art.

As used herein, the recitation of a numerical range for a variable is intended to convey that the embodiments may be practiced using any of the values within that range, including the bounds of the range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≧0 and ≦2 if the variable is inherently continuous. Finally, the variable can take multiple values in the range, including any sub-range of values within the cited range.

As used herein, unless specifically indicated otherwise, the word “or” is used in the inclusive sense of “and/or” and not the exclusive sense of “either/or.”

While only certain embodiments have been described, it will be understood by those skilled in the art that various changes in form and details may be made therein without departing from the spirit and scope as defined by the appended claims. Those skilled in the art will recognize, or be able to ascertain using no more than routine experimentation, many equivalents to the specific embodiments described specifically herein. Such equivalents are intended to be encompassed in the scope of the appended claims.

What is claimed is:

1. A device for treating a tissue with acoustic energy, comprising:
a source of electromagnetic energy;
an energy absorption medium configured to accept electromagnetic energy generated by the source;
a transduction member configured to transduce electromagnetic energy to acoustic energy and further configured to receive energy from the absorption medium and transduce the energy into acoustic energy;
a focusing element in communication with the transduction member and configured to focus the acoustic energy and direct the acoustic energy to a tissue during operation.

2. The device of claim 1, wherein the transduction member includes a liquid substance configured to convert optical energy to ultrasound.

3. The device of claim 1, wherein the transduction member includes a solid member configured to convert the electromagnetic energy to ultrasound.

4. The device of claim 1, wherein the transduction member further includes a piezoelectric element.

5. The device of claim 1, wherein the focusing element is a concave surface configured to transmit the acoustic energy.

6. A device for treating a tissue with acoustic energy, comprising:
a source of electromagnetic energy;
a converter configured to receive electromagnetic energy from the source and convert the electromagnetic energy to acoustic energy;
an output in communication with the converter and configured to transmit the acoustic energy to a tissue.

7. The device of claim 6, wherein the acoustic energy is ultrasound.

8. The device of claim 6, wherein the converter includes a piezoelectric element.

9. The device of claim 8, wherein the piezoelectric element is configured to focus the acoustic energy.

10. The device of claim 6, wherein the converter includes an array of piezoelectric elements.

11. The device of claim 10, wherein the array of piezoelectric elements are configured to focus the acoustic energy.

12. The device of claim 6, further comprising a focusing element configured to focus the acoustic energy.

13. The device of claim 6, wherein the converter further includes a focusing element configured to focus the acoustic energy.

14. The device of claim 6, wherein the output further includes a focusing element configured to focus the acoustic energy.

15. A device for treating a tissue with ultrasound energy, comprising:
a source of optical radiation;
an absorbing member configured to absorb energy from the source a converter configured to receive the absorbed energy from the absorbing member and to convert the energy to focused ultrasonic;
an output in communication with the converter and configured to deliver the ultrasonic energy to a tissue.

16. A method of treating tissue with acoustic energy, comprising:
irradiating a fist medium with electromagnetic radiation;
converting the electromagnetic radiation to acoustic energy;
focusing the acoustic energy; and
delivering the acoustic energy to a tissue to be treated.

17. The method of claim 16, wherein the acoustic energy is ultrasound.

18. The method of claim 16, wherein the step of focusing further comprises focusing the acoustic energy into a single beam.

19. The method of claim 16, wherein the step of focusing further comprises focusing the acoustic energy into an array of beams.

20. The method of claim 16, wherein the step of delivering further comprises delivering the acoustic energy to a portion of the tissue that selectively absorbs the acoustic energy.

21. A method of treating tissue with acoustic energy, comprising:
irradiating a fist medium with electromagnetic radiation;
converting the electromagnetic radiation to acoustic energy having at least one predetermined frequency component that is resonant with a structure in a tissue to be treated; and
delivering the acoustic energy to the tissue to be treated.

22. The method of claim 21, wherein the structure in the tissue is a portion of a hair.

23. The method of claim 21, wherein the structure in the tissue is at least a portion of a hair follicle.

24. The method of claim 21, further comprising focusing the acoustic energy.

25. The method of claim 21, wherein the acoustic energy is ultrasound.
